(19) United States
(12) Patent Application Publication
(10) Pub. No.: US 2013/0178789 A1
(43) Pub. Date: Jul. 11, 2013

(54) Monitoring Thermal Conditions To Vary Operation of an Ultrasonic Needle Tip of a Surgical Instrument

(71) Applicant: Novartis AG, Basal (CH)

(72) Inventor: Richard James Mackool, Greenwich, CT (US)

(73) Assignee: NOVARTIS AG, Basal (CH)

(21) Appl. No.: 13/764,815

(22) Filed: Feb. 12, 2013

Related U.S. Application Data
(63) Continuation-in-part of application No. 12/350,294, filed on Jan. 8, 2009, now Pat. No. 8,423,126.
Application No. 12/350,294 is a continuation of application No. 10/353,431, filed on Jan. 29, 2003, now abandoned.

(51) Int. Cl. A61F 9/007

(52) U.S. Cl. CPC A61F 9/00745
USPC 604/22

(57) ABSTRACT

A method and apparatus to operate a surgical instrument in response to a thermal condition being detected that warrants curtailment of further operation. When the thermal condition is reached, command signals are generated that cause a needle of the surgical instrument to either have its vibrational speed slowed, have its vibrational movement stopped, or have it withdrawn from its relative position. The detection is of infrared radiation wavelengths and is carried out with either a thermal imaging device or a thermal recognition device. A corresponding temperature of the detected infrared radiation wavelengths is compared to a critical temperature to determine whether the thermal condition has been reached.

### CROSS-REFERENCE TO COPENDING PATENT APPLICATIONS

This is a continuation-in-part of U.S. patent application Ser. No. 12/350,294 filed Jan. 8, 2009, which is a continuation of U.S. patent application Ser. No. 10/353,431 filed Jan. 29, 2003.

### BACKGROUND OF THE INVENTION

1. Field of the Invention

The present application relates to slowing or stopping the vibratory speed of an ultrasonic needle tip of a surgical instrument during ocular surgery or to withdrawing the needle, depending upon whether a medically unsafe thermal condition is likely to be reached if the ultrasonic needle tip is permitted to continue to vibrate at the same rate of speed and at the same location.

2. Description of Related Art

U.S. Pat. No. 5,409,481 describes a laser tissue welding control that includes monitoring through a surgical microscope and using an infrared radiation wavelength detector, i.e., a pyrometer, to ascertain temperature of a viewed object because the sensed infrared wavelengths are proportional to the temperature of the viewed object. An example of a pyrometer is that of Model M67S produced by Mikron Instrument Co.

U.S. Pat. No. 5,505,693 (the '693 patent) describes an invention that reduces heat generation during ocular surgery from giving rise to an medically unsafe thermal condition caused by heat generation from frictional effects of a vibratory motion of an ultrasonic needle tip of a surgical instrument acting on surrounding tissue. The normal temperature of body tissues is 37.degree. C., the surface tissue of the eye is normally slightly cooler, typically 35.degree. C., and a temperature of approximately 55.degree. C. or greater can cause damage to ocular tissue.

In a worst-case scenario of an ultrasonic transducer, driver and needle with a mass of 23 grams, a frequency of 60 KHz and a stroke length of 0.004 inches, the following calculation can be made. Ultrasonic power is approximately 32 Joules/second. If 80% of this energy is dissipated on the sleeve(s), the heat energy released would be 6 calories/second. Assuming that the area of a sleeve in contact with the tissue is 15-20 square millimeters and that a 3 mm thick region of tissue surrounding the sleeve accepts all the heat, temperature rise (in this region of tissue) would be 10.degree.-14.degree. C./second. Within this region of tissue and fluids, there will exist a temperature gradient, with the tissue in direct contact with the sleeve having the highest temperatures, and that most separated from direct sleeve contact experiencing lesser temperature elevations.

Under these circumstances, the approximate 55.degree. C. or greater limit would be reached in 1.5-2.0 seconds of full-power application by the ultrasonic transducer. In accordance with the invention, a dynamic friction coefficient of 0.1 between the outer needle surface and the inner sleeve surface will reduce heat generation by 90%, and will allow at least 15-20 seconds of operation before a tissue temperature of approximately 55.degree. C. or greater limit is reached.

It would be desirable to prevent a medically unsafe thermal condition from being reached while operating a vibratory needle of a surgical instrument.

### SUMMARY OF THE INVENTION

One aspect of the invention resides in detecting an infrared radiation wavelength with a thermal imaging or thermal recognition source, evaluating whether a critical temperature has been reached based on the detecting, and, if so, generating appropriate command signals to either slow or stop the needle vibratory speed or withdraw the needle from its relative position.

Another aspect of the invention resides in carrying out the detecting of infrared radiation wavelengths at a location along the surgical instrument other than at the needle tip, such as proximal to the needle tip, at the needle hub or at the needle driver. The surgical instrument is a conventional hollow needle used in phacoemulsification.

The conventional hollow needle used to carry out phacoemulsification has a shaft that terminates into the needle tip at the distal end and has a hub that is wider than the shaft and that is spaced from the needle tip by at least a portion of the shaft. The needle driver is adjacent the hub. A further aspect resides in carrying out the detecting of infrared radiation wavelengths at a location along the surgical instrument at a region of the needle shaft where there is no tissue interface, such as adjacent or proximal the hub yet spaced away from the needle tip and clear of a remaining portion of the shaft that penetrates tissue where there well may be a tissue interface present.

### BRIEF DESCRIPTION OF THE DRAWING

For a better understanding of the present invention, reference is made to the following description and accompanying drawings, while the scope of the invention is set forth in the appended claims:

FIG. 1 is a schematic representation of a thermal imaging or thermal recognition source, surgical operating microscope and surgical instrument in accordance with the invention.

FIG. 2 is a schematic representation as in FIG. 1 but of a further embodiment.

FIG. 3 is an isometric view of a conventional phacoemulsification needle in accordance with US Patent Application Publication No. US2005/0059939.

FIG. 4 is a sectional view of a conventional phacoemulsification needle of FIG. 3.

FIG. 5 is a top planar view of a conventional phacoemulsification handpiece being used in cataract removal in accordance with U.S. Pat. No. 3,589,363.

FIG. 6 is a cross-sectional view of a tip end of the handpiece in FIG. 5.

FIG. 7 is an enlarged view of the tip end of FIG. 6.

FIG. 8 is a planar view of an operative tip of FIG. 6.

### DETAILED DESCRIPTION OF THE INVENTION

The subject matter of the '693 patent is incorporated by reference. The present invention broadens and amplifies that subject matter with respect to providing infrared radiation wavelength detection, evaluating same with respect to a critical temperature, and issuing appropriate command signals.

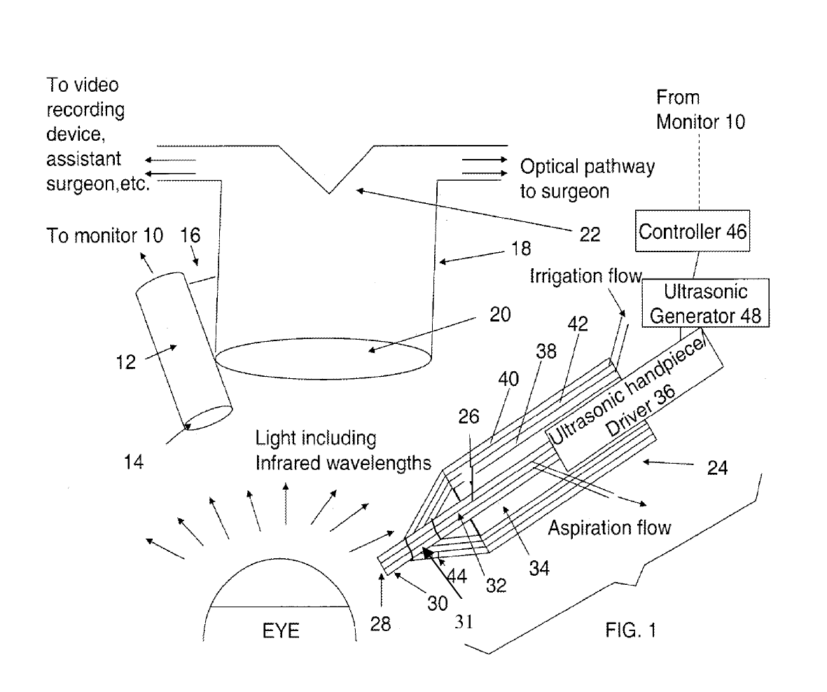

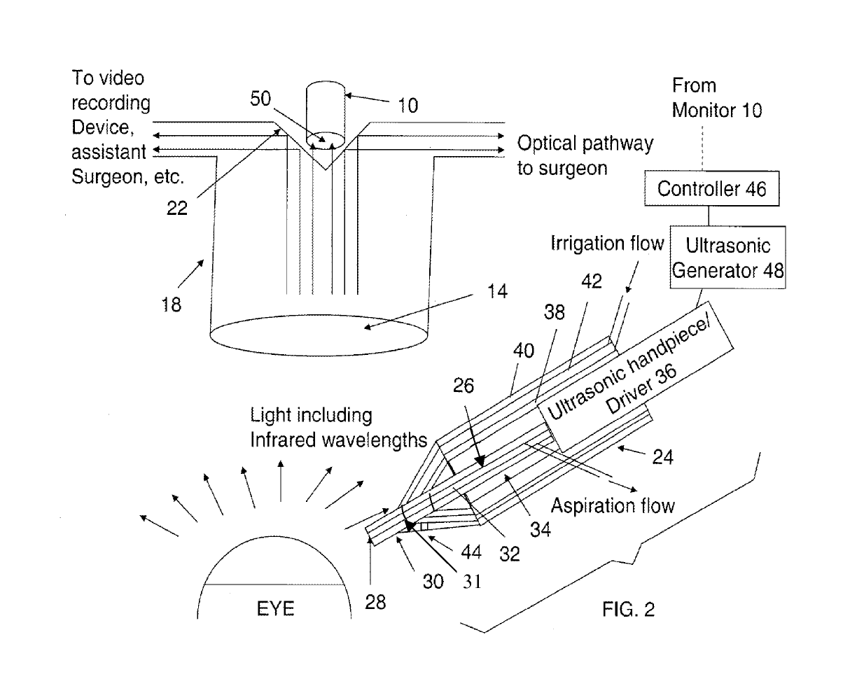

Turning to FIGS. 1 and 2, two embodiments are depicted. Each shows a monitor 10, a surgical operating microscope 18 and a surgical instrument 24. They differ with respect to the location of the monitor 10 and the components that direct the light, including infrared wavelengths, from a heat source to be monitored such as a surgical field in the eye. The monitor 10 may be a thermal imaging or thermal recognition source, such as a conventional thermal imaging camera or optical pyrometer, which is exemplified in U.S. Pat. No. 5,409,481, whose contents are incorporated by reference.

Turning to FIG. 1, the monitor 10 may detect the infrared wavelengths passing through an attachment tube 12. The attachment tube 12 has a lens 14 that collimates the light entering the tube 12. The tube 12 is attached via conventional fasteners 16 to the surgical operating microscope 18 so as to be aimed at the same location as that of the surgical operating microscope 18. The light travels through the tube 12 to reach the monitor 10, which may be an optical pyrometer or thermal imaging camera. The monitor 10 may be equipped with its own further lens (not shown) to focus the collimated light emerging from the tube 12.

In a conventional manner, the surgical operating microscope 18 has a lens 20 that collimates the light that passes through. Such light reflects off an optical beam splitter 22, which splits and directs the light in two directions; along an optical pathway to the surgeon and along an optical pathway to a conventional video recording device.

Turning to FIG. 2, the beam splitter 22 may be configured to allow the light to reflect, as in the embodiment of FIG. 1, and pass through the beam splitter 22 to reach the monitor 10. Alternatively, the monitor may be arranged within the optical pathway to the surgeon or to the video recording device. Indeed, an additional beam splitter may be arranged in such an optical pathway to reflect the infrared radiation wavelengths to the monitor 10 in a manner as described in U.S. Pat. No. 5,409,481, whose contents are incorporated by reference.

As concerns the embodiments of both FIGS. 1 and 2, the monitor 10 is automatically/desirably directed at the location where the heat arises due to the surgical instrument operation. That is, whenever the microscope is aimed at the surgical instrument, the attachment 12 is aimed in a like manner in unison with that of the surgical instrument.

If the monitoring is done through the optical channels or pathways of the microscope 18 as in the embodiment of FIG. 2, the objective lens(es) 14 of the microscope may need to be modified to be more transmissive of the infrared wavelengths that must be monitored. For example, the typical glass or fused silica used in microscopes might need to be changed to a quartz or other material known to transmit infrared radiation well.

The contents of U.S. Pat. No. 5,409,481 are incorporated herein by reference with respect to an infrared radiation detector or pyrometer used in conjunction with the surgical microscope to view and monitor an object whose temperature is to be ascertained. Also, the optical beam splitters of the present invention may be any conventional type such as that disclosed in U.S. Pat. No. 5,409,481 that is incorporated by reference.

The detected infrared wavelengths are proportional to a corresponding temperature that is compared to the critical temperature or critical change in the temperature. The critical temperature or critical change in temperature may constitute a demarcation of temperature ranges between those below that are medically safe for tissue to achieve and those above that risk and therefore being medically unsafe for tissue to achieve. When such a critical temperature or critical change in temperature is reached, an evaluation of such a thermal condition is made so that appropriate command signals may be generated by a controller 46 to a ultrasonic handpiece containing the needle driver 36 of a surgical instrument 24.

A conventional surgical instrument 24 to effect phacoemulsification is shown in FIGS. 1 and 2. It includes a needle shaft 26 having a suction port 28 at its tip 30, a portion 31 proximal to the tip 30. There is an aspiration flow passage 32 that constitutes the “hollow” of the hollow needle and is within the needle shaft 26. The needle shaft 26 enters a needle hub 34, which is wider than the needle shaft 26. The aspiration flow passage 32 permits an aspiration flow from the suction port 28 to a discharge, which may be attached to a suction device (not shown) such as a vacuum. The needle shaft 26 terminates into the needle tip 30 at the distal end. The needle hub 34 is wider than the needle shaft 26 and is spaced from the needle tip 30 by at least a portion of the needle shaft 26. The needle driver 36 is adjacent the needle hub 34.

The needle shaft 26 is driven to vibrate at ultrasonic speeds in a conventional manner by a surgical handpiece/driver 36 so as to break up tissue (such as cataract) to be suctioned through the aspiration flow passage 32. An optional elongated inner sleeve 38 and an optional elongated outer sleeve 40 are concentrically arranged about the needle shaft 26 to extend along the length of the needle shaft 26, although such sleeves are not required for practicing the present invention. A gap 42 is formed between the inner and outer sleeves 38, 40 to allow for an irrigation flow to emerge through one or more of the irrigation ports 44. The surgical handpiece/driver 36 responds to command signals from an ultrasonic generator 48 to drive the needle shaft 26 at a particular speed, such as ultrasonic, or to slow down or stop altogether. The monitor 10 may have a focusing lens 50 to focus the collimated light from the microscope lens 14 as shown in FIG. 2 or may be attached to the attachment tube 12 of FIG. 1.

The ultrasonic generator 48 receives direction form a controller 46, which evaluates sensed detection signals from the monitor 10 to determine whether the needle needs to be slowed, stopped or withdrawn based on the sensed detection signals and issue appropriate command signals to the ultrasonic generator 48 to drive the needle shaft 26 accordingly. If desired, two or more critical temperatures may be used to compare with the temperature corresponding to the sensed infrared radiation wavelengths. If the corresponding temperature matches the lower critical temperature(s), the speed of vibration of the needle would be slowed. If the corresponding temperature matches the highest critical temperature, either the needle would need to be stopped from vibrating or be withdrawn.

The monitor 10 may be configured to send the detection signals electronically to the controller 46 and/or audibly and/or visually to the physician to signify the thermal condition sensed. The controller 46 may respond by emitting detection signals that are indicative of the thermal condition sensed. Alternatively, the controller 46 may respond to an absence of signal generation as signifying that the thermal condition has been achieved.

Alternatively, the critical temperature may be a temperature sufficiently close to the medically unsafe temperature that prompt cessation or lowering of the amount of heat generation will avoid actually reaching the medically unsafe temperature, but the critical temperature is still at a medically safe level for surrounding tissue.

If the detected infrared wavelengths are correlated to a change in temperature, then by comparing this change in temperature with a critical change in temperature (corresponding to attaining the afore-mentioned critical temperature), a signal would generate that signifies that such a critical change in temperature had been achieved in a manner that is the same as for the previous discussion concerning achievement of the critical temperature.

The '693 patent calls for monitoring the temperature on the outer surf ace of the ultrasonic needle. However, I have conducted experimentation that indicates that the greatest temperature elevation may actually occur proximal to the tip of the needle shaft 26, such as along its hub 34 or even in the ultrasonic handpiece/metallic “driver” 36 to which the needle is attached (the ultrasonic handpiece/driver 36 is in turn connected to the ultrasonically vibratable material which is either a piezoelectric crystal or metallic, i.e., nickel).

The elevated temperature in either the ultrasonic handpiece/driver 36 or the needle hub 34 can rapidly spread to the needle tip 30 (and thus to the tissues of the eye that surround the needle tip 30). Therefore, monitoring the former areas should be done and the operation of the surgical instrument 24 should be modified to respond to temperature elevation in these regions (or even more simply to respond to the greatest temperature detected at any location within the surgical field) by discontinuing the ultrasonic vibration if a certain critical temperature is reached.

The concept is to monitor the temperature throughout the operating field, and to react, i.e. issue a warning and/or discontinue function(s) that are capable of increasing the temperature within the field. The location of the temperature elevation is of less importance than the temperature itself, although the greatest temperatures would almost certainly be achieved in an area in which tissue was in contact with the shaft (not the tip) or much more likely the sleeve surrounding the shaft (because the vast majority of procedures are performed with such a sleeve surrounding the shaft). In practice, the surgical instrument should be automatically disabled if a presettable temperature limit were achieved anywhere within the surgical field. This would never be achieved at the instrument tip because the tip is immersed in a cooling fluid within the eye.

The area at the tip, however, should still be monitored simply because there is no need to devise an expensive apparatus to detect the tip and then avoid monitoring it. Further, tissue interaction with the sleeve surrounding the needle shaft, or tissue interaction directly with the shaft itself if there is no sleeve, are the critical sites to be monitored.

Temperature elevation can theoretically originate at either the interface of the infusion sleeve with the needle, or with the infusion sleeve at the tissue. It is quite likely that it nearly always originates at the former, because the needle will almost if not always vibrate at a more rapid speed that the infusion sleeve (if the two vibrated together, a phenomenon known as “coupling”, then it is possible for the sleeve-tissue interface to be the site of frictional heat development).

So it is therefore most likely the case that it is when the needle presses the infusion sleeve against the surrounding tissue and the resultant lack of space between the needle and the motionless sleeve then causes friction to occur between the vibrating needle and the motionless inner surface of the sleeve that friction-induced heat develops. It of course spreads by simple convection through the sleeve and to the tissue that surrounds the outer surface of the sleeve.

An earlier one of my patents, namely U.S. Pat. No. 5,505,693 whose contents are incorporated herein by reference, covers the creation of a low coefficient of friction inner lining of the infusion sleeve to counter this problem. Thus, two sleeves are concentrically arranged about the needle shaft in accordance with U.S. Pat. No. 5,505,693 and as may be applied to the present invention. The frictional interface in that case may be between the vibrating needle shaft and the inner sleeve, between the two sleeves, and between the outer sleeve and surrounding tissue.

However, if no sleeve is present, then the frictional interface is obviously between the vibrating needle and the motionless tissue that surrounds it. However, less than 1% of phacoemulsification procedures are currently performed with a sleeveless vibrating needle. In such cases, infusion fluid is delivered into the eye through a needle inserted through a separate incision.

The surgical instrument includes an optional outer sleeve 40, an optional inner sleeve 38 and a gap 42 between the outer and inner sleeves 38, 40 through which irrigation flow is directed to emerge through an irrigation port 44 in the vicinity of the surgical field close to needle tip 30. The needle tip 30 has a suction port 28 to create an aspiration flow through an aspiration passage 32 from the surgical field to an aspiration port for removal of cataract tissue or the like.

The controller 46 generates command signals to the ultrasonic handpiece/driver 36 to fix the “stroke length” and thus speed of vibration of the needle shaft 26, such as in accordance with a pre-set program. The needle shaft 26 may vibrate at ultrasonic speeds. The controller 46 also generates command signals when warranted to slow or stop the needle shaft 26 and the ultrasonic handpiece/driver 36 responds accordingly to carry out the instructions to slow (reduce the “stroke length”) or stop the needle vibration. Phaco power is the ability of the phaco handpiece to cut or emulsify cataract. Phaco power is directly related to stroke length, frequency and efficiency of the handpiece. The stroke length is the distance by which a titanium phaco tip moves to and fro. The stroke length can be altered by changing the phaco power setting of the machine. Frequency is the number of times the tip moves and is fixed for a particular phaco handpiece.

Instead of issuing command signals to slow or stop the needle, the needle tip 30 may simply be withdrawn from its position so it no longer creates any friction with surrounding tissue that further heats the area. Such withdrawal may be done in one of two ways. One way is done manually by the surgeon who receives an indication that the detected temperature has reached a critical temperature. Upon learning of this thermal condition, the surgeon then withdraws the surgical instrument 24 so that the tip 30 does not continue to rub against tissue that results in frictional heating effects. Another method would utilize a needle that is retractable from its extended position where its tip 30 protrudes outward from the surgical instrument 24 to a retracted position where the tip 30 is withdrawn into the surgical instrument 24, the controller 46 may generate command signals to an appropriate device that may cause the ultrasonic handpiece/driver 36 to retract the needle even though the needle may or may not continue to vibrate at its set speed.

If the ultrasonic handpiece/driver 36 is incapable of retracting the needle shaft 26, the needle shaft 26 may be spring biased (not shown) into a retracted position such that its tip no longer protrudes. Command signals from the controller 46 may trigger a latch mechanism (not shown) to release the spring and thereby allow the needle to retract. To restore the needle to its extended position, the needle would be pushed, for instance manually, against the spring bias until latched into its extended position.

Once the needle speed is stopped due to the attainment of a thermal condition corresponding to the critical temperature condition, irrigating the surgical site cools it within seconds sufficiently to lower the temperature. The drop in temperature may be monitored until it reaches a sufficiently low temperature that the needle may resume vibration without risk of again achieving the thermal condition too quickly so that the physician is unable to work on the surgical site for a medically desired time period. If desired, the lower temperature that must be achieved before the needle will be permitted to vibrate again may be that of the original starting temperature before the temperature rose to attain the thermal condition corresponding to that of the critical temperature condition.

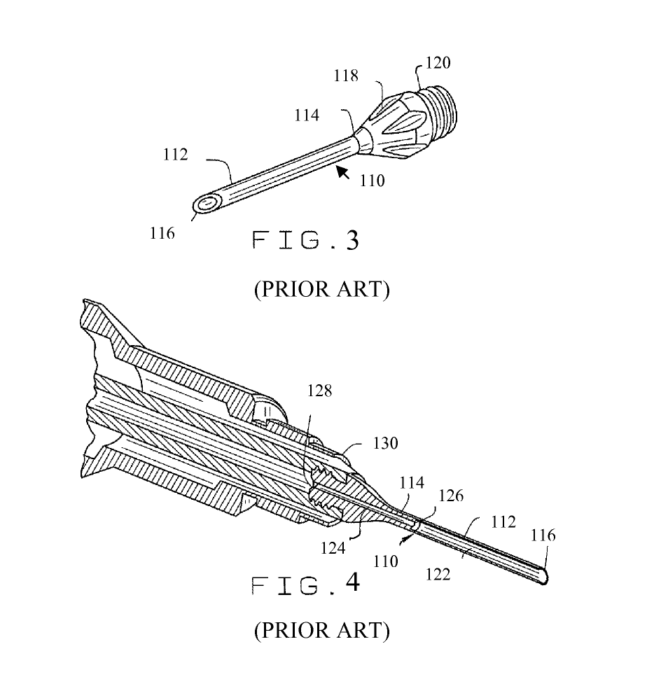

Turning to FIGS. 3 and 4, a conventional phacoemulsification instrument or phaco cannula 110 is shown in accordance with FIGS. 1 and 2 of US Patent Application Publication No. US 2005/0059939 A1, whose contents are incorporated herein by reference. Such a phaco cannula includes a needle 112 having a proximal end 114 and a distal end 116. Proximal end 114 is attached to a hub 118, which includes threads 120 for engagement with a surgical instrument shown below. Attachment structures other than threads 120 may be used to attach the phaco cannula 110 to a surgical instrument.

FIG. 3 shows a cut away perspective view of the conventional phacoemulsification surgical instrument or phaco canula 110. As can be seen, the first inner diameter 122 of the needle is larger than the second inner diameter 124 of the needle. In addition, a transition 126 from the first inner diameter 122 to the second inner diameter 124 is closer to the proximal end 114 than to the distal end 116. Another way to view cannula 110 is that it has an elongated needle 112 having a distal end 116 and a proximal end 128 structured for attachment to a surgical instrument 130, such as by threads 120 as shown in FIG. 4. In this view, the transition 126 is closer to proximal end 128 than to distal end 116.

FIGS. 1 and 2 show the conventional phacoemulsification surgical instrument 24 (e.g., handpiece) represented schematically. The structure of such a conventional phacoemulsification surgical instruments is exemplified by the conventional phacoemsulsification instrument 110 of FIGS. 3 and 4 of US Patent Application Publication No. US 2005/0059939 A1, whose contents are incorporated herein by reference.

Another conventional phacoemulsification instrument is that of U.S. Pat. No. 5,718,676, whose contents are incorporated herein by reference. With respect to FIG. 1 of U.S. Pat. No. 5,718,676, a previously known phaco-emulsification needle is shown comprising a hub and a hollow shaft extending from hub. A threaded portion extends away from hub on the proximal portion of shaft, as is conventional. Shaft terminates in tip at the distal end (i.e., remote from hub). The needle contains a central axially extending lumen through which material can be drawn from an eye using known techniques. A sleeve, which typically comprises a soft silicone material, is disposed over needle to form an annulus through which liquid can be supplied to the anterior chamber of the eye during aspiration. The sleeve can be compressed by the eye, and in particular, the portion surrounding the entry wound, to reduce liquid flow to an unacceptable extent.

Still another conventional phacoemulsification instrument is that of U.S. Pat. No. 6,402,769 B1, whose contents are incorporated herein by reference. One embodiment of a handpiece suitable for use has cutting tip, handpiece shell, ultrasound horn, torsional ultrasound crystals and longitudinal ultrasound crystals. The horn is held within the shell by an isolator. Crystals are held within the shell and in contact with the horn by a back cylinder and a bolt. The crystals vibrate ultrasonically in response to a signal generated by an ultrasound generator. The crystals are polarized to produce torsional motion. The crystals are polarized to produce longitudinal motion. The reference to “shell” in this patent may be considered to be that of the “hub”.

Ultrasonic handpieces and cutting tips are more fully described in U.S. Pat. Nos. 3,589,363; 4,223,676; 4,246,902; 4,493,694; 4,515,583; 4,589,415; 4,609,368; 4,869,715; and 4,922,902, the entire contents of which are incorporated herein by reference.

Yet another conventional phacoemulsfication instrument is that of U.S. Pat. No. 5,676,646, whose contents are incorporated herein by reference. That patent describes the hub as including a threaded portion that allows the cutting tip to be attached to an ultrasonic horn. The hub also includes wrenching flats that permit a wrench to engage cutting tip. Suitable wrenches are more fully described in U.S. Pat. No. Des. 351,095, the entire contents of which is incorporated herein by reference. The hub preferably has an overall diameter of between 0.100 inches and 0.150 inches with 0.140 inches being most preferred. The hub also contains a pair of asymmetric, hydrodynamic channels extending from flats. Channels preferably are cut at an angle of 45 degrees relative to the longitudinal axis of cutting tip, but the channels may be of any suitable number, size or shape as may be required to produce the desired tightening or untightening effect. The “cutting tip” in this patent is the same as the “needle tip”.

In use, as the cutting tip vibrates in a liquid medium, the hydrodynamic forces acting on channels vary as cutting tip moves forward and backward. As cutting tip moves forward, the increased hydrodynamic forces on channels tend to rotate hub clockwise. As the cutting tip moves backward, the reduced pressure around channels tend to rotated hub counterclockwise. The net result, however, is an overall clockwise turning of hub, because negative pressure exerted on channels on the backward stroke can never fall below negative 1 bar relative to atmosphere but increased pressure exerted on channels on the forward stroke can elevate to very high levels.

What is clear from these as well as all conventional phacoemulsification instruments is that the needle tip is not part of the hub and the needle tip is not proximal to the hub. Instead, there is a shaft that extends from the hub to terminate into the tip so a portion of the shaft is between the tip and the hub. Merriam Webster's medical dictionary defines “hub” as an enlarged base by which a hollow needle may be attached to a device (as a syringe).

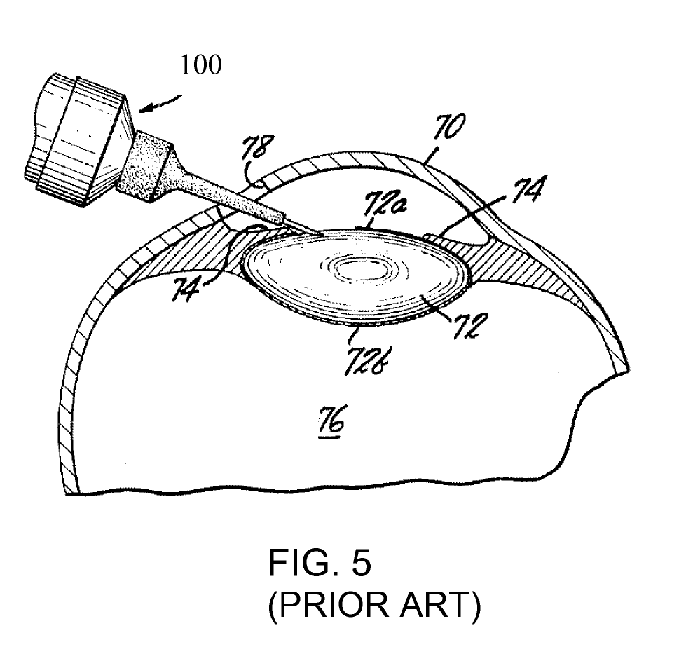

Turning to FIGS. 5-7, still another conventional handpiece is shown, which is in accordance with U.S. Pat. No. 3,589,363. Substantially all of the operative parts of the handpiece are enclosed within a casing indicated at 140, which includes a generally tubular barrel section 40a, a smaller diameter extension section 40b threaded onto the forward end of the barrel 40a, a forward end cap 40c having a small diameter tubular portion extending therefrom (see FIG. 3A) threadedly engaging the forward end of extension 40b, and a rear closure 40d threadedly engaging the rear portion of the barrel 40a. Preferably, all of the portions of the casing 140 are made of a metal such as stainless steel, which is sterilizable and which also provides shielding for the electrical and magnetic components within the handpiece.

The vibratory assembly contained within the casing 140 is composed of a transducer portion 142 and a connecting body 146, the latter preferably in the form of an acoustic impedance transformer. The transducer element 142 may be of any suitable type capable of converting high frequency alternating current signals into corresponding longitudinal mechanism vibrations. In the embodiment illustrated, the transducer is composed of a stack of thin sheets of magnetostrictive material such as nickel, Permendur, or other similar material, insulated from each other and firmly secured together such as by brazing at the ends. As shown, the stack is divided by a lengthwise elongated opening effectively separating the stack into two separate vibratory sections, with the coil 144 wound about each section or leg separately, in such fashion as to produce in phase vibration in both legs.

The forward end of the stack 142 is coupled, such as by a threaded connection as shown or by a permanent bond, to the input end of the connecting body 146. Preferably, a washer 43 is provided between the end of the stack and the input surface of the member 46 to render the transmission of vibratory energy to the member 146 more effective.

As indicated above, the connecting body 146 preferably is in the form of an acoustic impedance transformer whereby the amplitude of the longitudinal vibrations induced in the stack 142 may be increased for application to the operative tip of the handpiece. For this purpose, the member 46 may be formed of a single piece of vibration transmitting material such as Monel metal, having a relatively massive input section 46a and a relatively slender output section 46b, with a tapered transition region 46c.

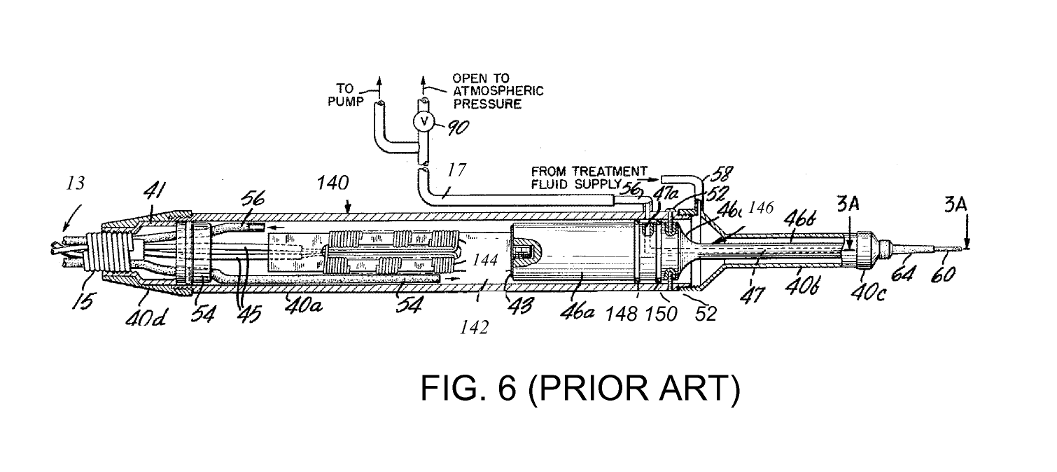

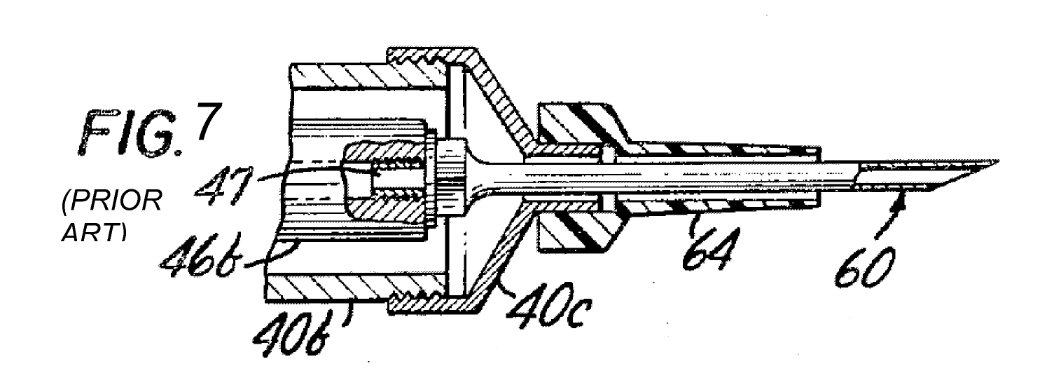

In FIG. 6, mounting means are provided in the form of a pair of resilient rings, generally referred to as “O” rings, located in a pair of spaced grooves extending circumferentially around the input section 46a of the transformer, as close as possible to the transition region where the nodal plane would be located. The rings 148 and 150 are of such diameter that they effect a fluidtight seal between the surface of the transformer input section 46a and the inner wall of the housing barrel 40a.

In addition to the spaced sealing rings 148 and 150, a plurality of screws 52 are provided angularly disposed about the axis of the casing, for the purpose of preventing longitudinal or rotational movement of the vibratory structure within the casing and also for radially centering the vibratory structure within the casing. By adjusting the several screws, concentricity of the interior elements of the handpiece and the casing sections may be obtained.

The sealing rings 148 and 150 divide the interior volume of the housing 40 into three independent fluid chambers. The ring 148 in conjunction with the end closure means to be described hereinafter will form a first chamber in which are disposed the magnetostrictive stack 142 and a portion of the transformer input section 46a. An annular chamber of relatively short axial dimension is formed between the two O-rings 148 and 150, and a third chamber is formed forwardly of the ring 150 including the free space within the casing extension 40b.

The rear portion of the casing barrel 140 is sealed off by means of a grommet 55 which is press fitted into the end of the barrel to form a watertight seal therewith. The grommet is provided with openings through which the electrical leads 45 pass from the conduit 13 to the coil 44. In addition, a coolant fluid inlet tube 54 passes through the grommet 52 and extends within the barrel 40a to a point adjacent the forward end of the stack 42. Fluid outlet tube 56 is also passed through the grommet 52 and into the conduit 13 along with the tube 54. The cooling water supply continually flows into the chamber enclosing the magnetostrictive element from the tube 54, and is withdrawn through the outlet tube 56 after passing over the heat producing elements. It will be understood that the leads 45 and conduits 54 and 56 pass through the grommet 52 in fluid tight relationship.

To provide strain relief for the conduit 13 and minimize entanglement, a wire coil 15 may be wrapped around the portion of the conduit 13 adjacent the handpiece, in place of the plastic tubing enclosing the remainder of the conduit. The coil 15 engages a helical groove provided internally of a retaining element 41. The latter element is compressed about the coil 15 and firmly retained against movement by the threaded cap 40d.

The impedance transformer 146 is provided with an axial bore 47 extending from the free or forward end of the output section 46b and into the input section 46a, to a point between the two sealing rings 148, 150. A radial bore 47a connects the bore 47 to the periphery of the transformer 146 and into the annular chamber between the sealing rings. Nipple 56 is connected to the periphery of the handpiece barrel 40a and is provided with an internal bore extending through the casing and communicating with the annular chamber between the rings 148 and 150 and thus with the bore 47 via the radial bore 47a.

A second nipple 58 is connected to the casing extension 40b at a point near its threaded coupling to the barrel 40a. The latter nipple includes an internal passage communicating with the annular chamber extending forwardly of the sealing ring 50 and including the space between the impedance transformer 146 and the inner walls of the casing sections. As shown best in FIG. 7, this latter chamber extends past the free end of the output section of the connecting body 46b and through the cap 40c.

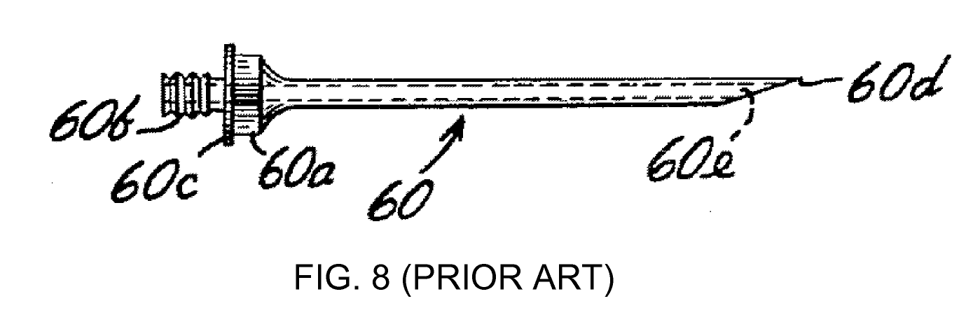

The operative tool or tip which actually comes into contact with the material to be broken apart and removed is designated by the numeral 60. Referring to FIG. 8, the tip 60 is elongated and provided with a thickened shank portion 60a which preferably is formed with at least a pair of flats to accommodate a wrench for tightening. A threaded connection portion 60d is formed integrally with the base portion 60a, and a washer 60c, of efficient vibration-transmitting material, is disposed adjacent the shoulder between the portion 60a and 60b. The other end of the tip 60 is shaped in a manner dependent upon the particular type of material or tissue to be broken apart and removed and the shape of the portion to be removed or its surrounding material. In FIG. 5A an acute-angled taper is provided to leave a relatively sharp, rounded edge 60d. An axial bore 60e extends completely through the tip 60 to provide a fluid passage from threaded end 60b to the outer or working end of the tip.

Referring back now to FIG. 7, the bore 47 in the transformer 146 is provided at the free end of the output section with internal threads adapted to receive the threaded portion 60b of the tip 60. To attach the tip 60, or to replace one already in position, the end cap 40c is threadedly disengaged from the casing extension 40b and slid backover and away from the tip 60. A small wrench may be used to engage the flats on the base section 60a to remove a tip already in place or to snugly insert a new tip. The end cap 40c is then replaced and the tool is assembled for use.

The operative tip 60, being firmly coupled to the output end of the impedance transformer 146, will be longitudinally vibrated thereby at the operating frequency and essentially with the amplitude available at the end of the output section 46b. The operative tip 60 preferably is formed of an extremely hard, sterilizable material, such as titanium, and for most surgical applications is made of extremely small dimension. For example, in the instrument as used for cataract removal operations, the operative tip had an outside diameter of approximately 1 millimeter. Since this is the only portion of the instrument that is brought into contact with the tissue to be broken apart and removed, it will be evident that only a very short incision needs to be made in the outer surface to permit access of the tip.

Where the material to be broken apart and removed is relatively deep below the surface, it is undesirable for the shank of the operating tip to be brought into contact with the surrounding tissue, especially if that tissue is healthy and not to be removed. Since the tip 60 is vibrating at a high frequency, heat will be developed due to the rubbing action and damage to delicate tissues can result.

To avoid this possibility, a sheath 64 of a strong, heat-resistant and inert material, such as the plastic known as “Teflon”, is provided. As shown best in FIG. 7, the sheath 64 is provided with an axial bore of a diameter somewhat greater than the outer diameter of the tip 60 and has a base section with a counterbore that snugly engages the tubular portion extending from the forward end of the end cap 40c. The fit between the latter two parts is made such that the sheath 64 may be secured to the end cap 40c with manual pressure but will not shake loose under normal usage.

The barrel of the sheath 64 extends along a considerable length of the operative tip 60 and has an outer diameter of sufficient thickness to provide the necessary structural rigidity. Preferably, it is slightly tapered, as shown. The annular clearance between the inner surface of the sheath 64 and the outer surface of the operative tip 60 serves as an extension of the fluid passage formed between the transformer section 46b and the casing extension 40b.

As will be explained in greater detail in connection with FIG. 6, alternating current electrical energy having a superimposed direct voltage bias thereon is coupled from the unit 12 and via conductors 45 to the coil on the transducer 142. The vibratory structure is thereby set into longitudinal vibration at the oscillator frequency, with the consequent vibration of the operative tip 60. For purposes of example, the amplitude of the alternating current supply may be set such that the working end of the tip 60 has a stroke amplitude of approximately 0.003 inch. At the same time, of course, a coolant supply is circulating in the chamber housing the transducer structure.

As the operative tip is brought into contact with the material to be broken apart and removed, treatment fluid from the supply 20 is provided through the conduit 22 and the nipple 58 to the passage formed between the connecting body 46 and the casing extension 40b and thence through the annular space between the cap 40c, sheath 64, and the tip 60. The tissue adjacent the operating tip is thereby bathed with the treatment fluid.

The treatment fluid serves two purposes. In addition to maintaining the operative tip relatively cool during use, thereby reducing possible harm to healthy tissue, it provides a dispersion medium in which particles of tissue are suspended as they are broken away from the tissue mass. It will of course be realized that the treatment fluid is being brought into direct contact with delicate tissue and accordingly must be of a neutral nature. In the case of cataract removal, for example, a balanced isotonic saline solution is suitable for this purpose.

Withdrawal of the suspension of the tissue particles in the treatment fluid is effected through the hollow operative tip, the bore 47 in the transformer 146, the connecting passage 47a and nipple 56, through the conduit 17 and to the withdrawal means, for example a pump 16. During the operative procedure, the volume of treatment fluid supply is controlled, along with the pump operation, so that a proper amount of treatment fluid is maintained at the operative site and overflow is minimized.

The use of the instrument 100 as applied to cataract removal is illustrated in FIG. 5. A portion of a simplified cross section of a human eye is shown to illustrate the manner in which the device is employed. The opaque lens or cataract which is to be broken apart and removed to is designated by the numeral 72 and is encased in a membrane including an outer portion 72a known as the anterior capsule and a rear portion 72b known as the posterior capsule. The iris is designated by the numeral 74 and the major gel-filled portion of the eye, or vitreous, is shown at 76. The cornea, the transparent outer surface of the eye, is shown at 70.

To avoid having to pierce or cut the iris, suitable drugs are administered to dilate the iris to its maximum extent, so that as much of the anterior capsule 72a is exposed as is possible. A small incision 78 is then made in the transparent cornea fluid as far as possible from the center of the pupil area. This incision need only be about 1 to 3 mm. in length to provide proper access for the operative tip of the vibratory assembly.

The anterior capsule 72a is penetrated, first, either by the operative tip of the vibrating assembly or with a surgical instrument. Once an opening in the anterior capsule has been made, such as indicated in FIG. 4, the operative tip is inserted into the body of the cataract 72, whereby the lens tissue mass is broken apart into minute particles. During this portion of the operation, the transducer is energized and the pump is activated to provide suction force at the operative tip, along with a supply of treatment fluid.

In the space of a few minutes, all of the cataract tissue 72 is broken apart and the particles, together with the fluid in which they are suspended, withdrawn by the instrument. Thereafter, the remnants of the anterior capsule and the posterior capsule are withdrawn with capsule forceps. This completes the cataract removal. The small incision 78 is subsequently sutured to conclude the surgical procedure. As compared to the conventional cataract removal, which requires a 180 degree incision around the cornea, trauma to the patient and recovery time are substantially reduced.

In FIG. 5, the operative tip of the vibratory assembly is illustrated as inserted into the eye with the plastic sheath 64 in place. As the surgeon maneuvers the instrument to reach all of the cataract tissue, any contact that occurs between the instrument and the other parts of the eye is on the sheath, which is not vibrating and therefore cannot damage any of the delicate tissue. In this instance, the sheath also serves to discharge the treatment fluid more directly at the operative site. It will be understood of course, that the sleeve 64 may be removed and the operative tip employed without it where operative conditions permit.

In the cataract removal procedure, the treatment fluid supply serves a purpose in addition to providing a dispersion medium for the particles of unwanted tissue and a coolant for the operative tip, by serving to maintain sufficient pressure within the anterior chamber of the eye, between the anterior capsule 72a and the cornea 70, whereby collapse of the latter is avoided.

Reference is made to the Boston School of Medicine website at http://www.bu.edu/eye/phacoprimer/phacoemulsification/. That website includes a video showing a phacoemulsification technique. There is a tissue interface with the needle tip and the portion of the needle shaft that extends from the needle hub. However, there is no tissue interface with the needle hub and no tissue interface with the ultrasonic driver.

FIGS. 1 and 2 include a schematic representations of a phacoemulsification handpiece, but the structure depicted is to be interpreted as being no different from the conventional handpieces of FIGS. 3-7. To the extent there is any discrepancy in the rendition of the conventional handpiece in FIGS. 1 and 2 with the conventional handpiece of FIGS. 3-7, the discrepancy is to be made consistent with that of FIGS. 3-7.

An embodiment of the present invention lies in apparatus to detect and respond to a thermal condition during phacoemulsification. The apparatus includes a surgical instrument operative to perform phacoemulsification. The surgical instrument includes a hollow needle and includes a driver operative to vibrate the hollow needle at a speed of vibration and including a suction operative to aspirate fluid through the hollow needle. The hollow needle has a shaft with the shaft terminating at a distal end into a tip. At least one hollow sleeve may be provided through which extends the shaft with the tip protruding out of the at least one hollow sleeve so as to give rise to an interface between the at least one sleeve and surrounding tissue during performance of the phacoemulsification. A thermal imaging or thermal recognition source is arranged to detect infrared radiation wavelengths that emanate from each of the shaft at locations away from the tip, the at least one hollow sleeve, an interface between the shaft and the at least one hollow sleeve, and the interface between the at least one hollow sleeve and surrounding tissue. Preferably, means is provided, such as a controller, for making a determination as to whether a thermal condition has been reached based upon a comparison of the detected infrared radiation wavelengths with criteria indicative of the thermal condition and for generating at least one signal in response to the determination being that the thermal condition has been reached.

In addition, means may be provided for slowing or stopping vibration of the needle in response to receipt of the at least one signal from the controller. Further means may be provided for triggering activation of an alarm in response to receipt of the at least one signal from the controller—the alarm may be visual, audible, vibratory and any combination thereof and of conventional construction. For instance, a visual alarm may be flashing lights or a blinking warning message in a display screen. The audible alarm may be the enunciation of a high pitched audible tone. The vibratory alarm may be vibrations imparted to a device in contact with the operator of the surgical instrument, such as a foot pedal. A combination of such alarms may be employed that commence at the same time or in succession.

While the foregoing description and drawings represent the preferred embodiments of the present invention, it will be understood that various changes and modifications may be made without departing from the spirit and scope of the present invention.

What is claimed is:

1. A method of detecting and responding to a thermal condition during phacoemulsification, comprising performing phacoemulsification with a surgical instrument operative to vibrate a hollow needle with a driver at a speed of vibration and to aspirate fluid under suction through the hollow needle, the hollow needle having a shaft, the shaft terminating at a distal end into a tip, at least one hollow sleeve through which extends the shaft with the tip protruding out of the at least one hollow sleeve so as to give rise to an interface between the shaft and the at least one sleeve and to give rise to a further interface between the at least one shaft and surrounding tissue during the performing of phacoemulsification; detecting infrared radiation wavelengths with a thermal imaging or thermal recognition source that emanate from each of the shaft, the at least one hollow sleeve at locations away from the tip, the interface between the shaft and the at least one hollow sleeve, and the further interface between the at least one hollow sleeve and surrounding tissue; making a determination as to whether a thermal condition has been reached based upon a comparison of the detected infrared radiation wavelengths with criteria indicative of the thermal condition; and generating at least one signal in response to the determination being that the thermal condition has been reached.

2. The method of claim 1, further comprising slowing or stopping vibration of the needle in response to the at least one signal.

3. The method of claim 1, further comprising driving the needle to vibrate at ultrasonic speeds; and triggering activation of an alarm in response to the at least one signal, the alarm being selected from a group consisting of visual, audible, vibratory and any combination thereof.

4. The method of claim 1, further comprising aiming a surgical operating microscope, and arranging the thermal imaging or thermal recognition source to make the detection of the infrared radiation wavelengths through the optical pathways of the surgical operating microscope.

5. The method of claim 1, further comprising attaching an optical attachment having an optical pathway to an outer housing of a surgical operating microscope and arranging the thermal imaging or thermal recognition source to make the detection through the optical pathways of the optical attachment.

6. An apparatus to detect and respond to a thermal condition during phacoemulsification, comprising a surgical instrument operative to perform phacoemulsification, the surgical instrument including a hollow needle and including a driver operative to vibrate the hollow needle at a speed of vibration and including a suction operative to aspirate fluid through the hollow needle, the hollow needle having a shaft, the shaft terminating at a distal end into a tip, at least one hollow sleeve through which extends the shaft with the tip protruding out of the at least one hollow sleeve so as to give rise to an interface between the shaft and the at least one sleeve and to a further interface between the at least one sleeve and surrounding tissue during performance of the phacoemulsification; a thermal imaging or thermal recognition source arranged to detect infrared radiation wavelengths that emanate from each of the shaft at locations away from the tip, the at least one hollow sleeve, the interface between the shaft and the at least one hollow sleeve, and the further interface between the at least one hollow sleeve and surrounding tissue; means for making a determination as to whether a thermal condition has been reached based upon a comparison of the detected infrared radiation wavelengths with criteria indicative of the thermal condition and for generating at least one signal in response to the determination being that the thermal condition has been reached.

7. The apparatus of claim 6, further comprising slowing or stopping vibration of the needle in response to the at least one signal.

8. The apparatus of claim 6, further comprising means for triggering activation of an alarm in response to the at least one signal, the alarm being selected from a group consisting of visual, audible, vibratory and any combination thereof.

9. The apparatus of claim 6, further comprising a surgical operating microscope, the thermal imaging or thermal recognition source being arranged to detect the infrared radiation wavelengths through optical pathways of the surgical operating microscope.

10. The apparatus of claim 6, further comprising an attachment having an optical pathway and that is attached to an outer housing of the surgical operating microscope, the thermal imaging or thermal recognition source being arranged to detect the infrared radiation wavelengths through an optical pathway of the attachment.

11. A method of detecting and responding to a thermal condition during phacoemulsification, comprising performing phacoemulsification with a surgical instrument operative to vibrate a hollow needle with a driver at a speed of vibration and to aspirate fluid under suction through the hollow needle, the hollow needle having a shaft, the shaft terminating at a distal end into a tip so as to give rise to an interface between the shaft and surrounding tissue during the performing of the phacoemulsfication; detecting infrared radiation wavelengths with a thermal imaging or thermal recognition source that emanate from each of the shaft at locations away from the tip and the interface between the shaft and surrounding tissue; making a determination as to whether a thermal condition has been reached based upon a comparison of the detected infrared radiation wavelengths with criteria indicative of the thermal condition; and generating at least one signal in response to the determination being that the thermal condition has been reached.

12. The method of claim 11, further comprising slowing or stopping vibration of the needle in response to the at least one signal.

13. The method of claim 11, further comprising triggering activation of an alarm in response to the at least one signal, the alarm being selected from a group consisting of visual, audible, vibratory and any combination thereof.

14. The method of claim 11, further comprising aiming a surgical operating microscope, and arranging the thermal imaging or thermal recognition source to make the detection of the infrared radiation wavelengths through the optical pathways of the surgical operating microscope.

15. The method of claim 11, further comprising attaching an optical attachment having an optical pathway to an outer housing of a surgical operating microscope and arranging the thermal imaging or thermal recognition source to make the detection through the optical pathways of the optical attachment.

16. An apparatus to detect and respond to a thermal condition during phacoemulsification, comprising a surgical instrument operative to perform phacoemulsification, the surgical instrument including a hollow needle and including a driver operative to vibrate the hollow needle at a speed of vibration and including a suction operative to aspirate fluid through the hollow needle, the hollow needle having a shaft, the shaft terminating at a distal end into a tip so as to give rise to an interface between the shaft and surrounding tissue during performance of the phacoemulsification; a thermal imaging or thermal recognition source arranged to detect infrared radiation wavelengths that emanate from each of the shaft at locations away from the tip and the interface between the shaft and surrounding tissue; means for making a determination as to whether a thermal condition has been reached based upon a comparison of the detected infrared radiation wavelengths with criteria indicative of the thermal condition; and means for generating at least one signal in response to the determination being that the thermal condition has been reached.

17. The apparatus of claim 16, further comprising means for slowing or stopping vibration of the needle in response to the at least one signal.

18. The apparatus of claim 16, means for triggering activation of an alarm in response to the at least one signal, the alarm being selected from a group consisting of visual, audible, vibratory and any combination thereof.

19. The apparatus of claim 16, further comprising a surgical operating microscope, the thermal imaging or thermal recognition source being arranged to detect the infrared radiation wavelengths through optical pathways of the surgical operating microscope.

20. The apparatus of claim 16, further comprising an attachment having an optical pathway and that is attached to an outer housing of the surgical operating microscope, the thermal imaging or thermal recognition source being arranged to detect the infrared radiation wavelengths through an optical pathway of the attachment.
